(19) United States
(12) Patent Application Publication
(10) Pub. No.: US 2018/0340149 A1
(43) Pub. Date: Nov. 29, 2018

(54) METHOD FOR CULTURING OSTEOCYTE

(71) Applicant: SHENZHEN EXCELLENT TECHNOLOGY LIMITED LIABILITY COMPANY, Guangdong (CN)

(72) Inventors: Xiaowen ZHAO, Guangdong (CN); Dongfeng ZHANG, Guangdong (CN)

(21) Appl. No.: 15/776,427

(22) PCT Filed: Jul. 19, 2016
(86) PCT No.: PCT/CN2016/090524

§ 371 Date: May 16, 2018

(30) Foreign Application Priority Data
Nov. 18, 2015 (CN) 201510797789.2

(51) Int. Cl. C12N 5/077; A61L 27/38; B33Y 10/00; B33Y 70/00; B33Y 80/00

(52) U.S. Cl. CPC C12N 5/0654; A61L 27/3821; A61L 2430/02; B33Y 70/00; B33Y 80/00; B33Y 10/00

(57) ABSTRACT

A method for culturing osteocytes includes: obtaining an original three dimensional image of a bone tissue of a patient by collecting original image data of the bone tissue; determining a bone nidus and obtaining reverse data for fitting the bone nidus with normal bone parts other than the bone nidus by analyzing and dissecting the original three dimensional image; obtaining a preliminary three dimensional bone tissue image by simulating and matching the bone nidus and the normal bone parts other than the bone nidus based on the reverse data repeatedly; obtaining a complete three dimensional bone tissue image by designing an internal configuration of the preliminary three dimensional bone tissue image according to a bone tissue database; and obtaining a bone tissue culturing structure by printing the complete three dimensional bone tissue image through a multidimensional printer.

### TECHNICAL FIELD

The present disclosure belongs to the field of macromolecular materials, and for example, relates to a method for culturing osteocytes.

### BACKGROUND

Tumor, inflammation, trauma and the like may cause bone defects in a human bone tissue. How to repair the bone defects is always a difficult problem in tissue engineering. A rapid development of bone tissue engineering in recent years provides another way for people to solve a treatment problem of large segmental bone defects. Especially in recent years, a development of technologies such as three-dimensional biodegradable material, cell culture and the like predicts a promising prospect of the bone tissue engineering. The bone tissue engineering is used for repairing bone tissue defects as follows. Isolated autologous high-concentration osteoblasts, bone marrow stromal stem cells or chondrocytes are cultured and amplified in vitro, and then are planted on a natural or artificial synthetic cell scaffold (also referred to as extracellular matrix (ECM)) having good biocompatibility and capable of being gradually degraded and absorbed by human bodies, which has a complex heterogeneous porous configuration to create a proper microenvironment for adhesion, growth, proliferation and osteogenesis of seed cells. Namely, such biological scaffold can provide a three-dimensional living space for the cells, is favorable for the cells to obtain sufficient nutriments to conduct gas exchange, and exclude waste material, so that these cells grow on a three-dimensional scaffold in a preset form. Then, hybrid material of these cells is implanted at a bone defect site. Therefore, the osteocytes implanted are continuously proliferated while the biomaterial is gradually degraded.

Many traditional methods can be used for preparing a three-dimensional structure having a microenvironment suitable for cell growth, such as fiber bonding, emulsion freeze-drying, solvent casting/particulate leaching, gas foaming, thermally induced phase separation and electrospinning. These methods need to apply a mold to or manually realize a shape of the scaffold. Since most of these methods are based on manual operations, such methods fail to fully play a role of computer software design and realize controllability for the porous configuration, are difficult to ensure interconnection between pores and are difficult to form gradient pores and material gradient structures. Therefore, the scaffolds prepared through such methods do not have consistent microstructures and microenvironments. Moreover, external structures of the scaffolds prepared through such methods do not coincide with anatomical structures of damaged tissues and organs of patients, and a forming cycle is long and the efficiency is low, causing that individualized manufacturing and production requirements for the scaffolds cannot be met.

### SUMMARY

Embodiments of the present disclosure provide a method for culturing osteocytes. According to embodiments of the present disclosure, seed cells and mutated stem cells grow and propagate in an osteocyte culturing structure produced by 3D printing, and form a bone tissue finally.

A method for culturing osteocytes includes:

obtaining an original three dimensional image of a bone tissue of a patient by collecting original image data of the bone tissue;

determining a bone nidus and obtaining reverse data for fitting the bone nidus with normal bone parts other than the bone nidus by analyzing and dissecting the original three dimensional image;

obtaining a preliminary three dimensional bone tissue image by simulating and matching the bone nidus and the normal bone parts other than the bone nidus based on the reverse data repeatedly;

obtaining a complete three dimensional bone tissue image by designing an internal configuration of the preliminary three dimensional bone tissue image according to a bone tissue database; and

obtaining a bone tissue culturing structure by printing the complete three dimensional bone tissue image through a multidimensional printer.

Optionally, the designing an internal configuration of the preliminary three dimensional bone tissue image includes: designing and adjusting the internal configuration of the preliminary three dimensional bone tissue image according to conditions required for osteocyte growth and propagation.

Optionally, the designing and adjusting the internal configuration of the preliminary three dimensional bone tissue image includes: designing the internal configuration of the preliminary three dimensional bone tissue image into a scaffold with a porous configuration; and adjusting porosity of the porous configuration, a size of aperture, a shape of a hole, surface area of the scaffold and a configuration of the scaffold.

Optionally, the shape of the hole in the porous configuration includes cylindrical or spherical shape.

Optionally, the method further includes the following steps after the obtaining a bone tissue culturing structure:

measuring and obtaining parameters of the bone tissue culturing structure; and

comparing the parameters of the bone tissue culturing structure with design parameters of the complete three dimensional bone tissue image to determine whether the bone tissue culturing structure meets design requirements.

Optionally, the parameters of the bone tissue culturing structure include at least one of the followings: a porosity of the internal configuration of the bone tissue culturing structure, a size of aperture, a shape of aperture, surface area of the scaffold and a configuration of the scaffold.

Optionally, the method further includes the following step after the obtaining a bone tissue culturing structure: comparing the bone tissue culturing structure with a physical model of the normal bone parts other than the bone nidus to perform verification.

Optionally, the method further includes the following step after the obtaining a bone tissue culturing structure: detecting whether the bone tissue culturing structure served as a microenvironment structural body meets conditions required for osteocyte growth and propagation.

Optionally, the bone tissue culturing structure is a physical structure matched with the normal bone parts other than the bone nidus.

Optionally, the method further includes the following step after the obtaining a preliminary three dimensional bone tissue image: converting or storing the preliminary three dimensional bone tissue image as a file of at least one of the following formats: stl, stp, obj, max, 3ds, ma, vtk and igs.

Optionally, the method further includes the following step after the obtaining a complete three dimensional bone tissue image: converting or storing the complete three dimensional bone tissue image as a file of at least one of the following formats: stl, stp, obj, max, 3ds, ma, vtk and igs.

Optionally, the multidimensional printer includes: a three dimensional printer, a four dimensional printer or a five dimensional printer.

The method for culturing osteocytes provided in embodiments of the present disclosure designs a bone tissue culturing structure suitable for osteocyte growth. The configuration of the bone tissue culturing structure is adjustable. Individual bone data (the normal bone parts other than the bone nidus of the patient) is matched with the data in the bone tissue database, and the bone tissue culturing structure and the patient can be subjected to individualized matching. The quantity, sizes, distribution and shapes of micropores in the internal configuration of the bone tissue culturing structure can be manually controlled. The osteocytes can be accurately positioned while the bone tissue culturing structure is built. The osteocytes can be contained on the surface and in the bone scaffold, thereby providing a possibility of realizing reasonable spatial distribution of the osteocytes; conduit structures similar to blood vessels in natural bone tissue can be built; and conduit distribution, conduit diameters and porosity can be manually controlled to facilitate exchange of oxygen, nutrients and metabolites between the osteocytes in the conduit tissue culturing structure and an external environment and establish a basis for further culturing and forming blood vessel structures and even finally producing corresponding physiological functions.

The bone tissue culturing structure suitable for osteocyte growth and propagation, which is printed by the multidimensional printer, has a set of mutually communicated channel systems, so that the cells and the nutrients are smoothly transported into the tissue and organ structure in a rebuilding process of the tissue and organ; and the cells are uniformly deposited into a microchannel.

The bone tissue culturing structure, which is prepared by the method for culturing osteocytes provided in embodiments of the present disclosure, is suitable for cell growth and propagation, and can provide a proper microenvironment for forming the bone tissue that remedies the bone nidus.

### BRIEF DESCRIPTION OF DRAWINGS

FIG. 1 is a flow chart illustrating a method for culturing osteocytes according to an embodiment of the present disclosure;

FIG. 2 is a schematic diagram illustrating an original configuration of a bone tissue of a patient according to an embodiment of the present disclosure; and

FIG. 3 is a schematic diagram in which a bone tissue culturing structure according to an embodiment of the present disclosure and remained normal parts of a bone tissue are matched with each other.

### DETAILED DESCRIPTION

The present disclosure is described below in combination with drawings and embodiments.

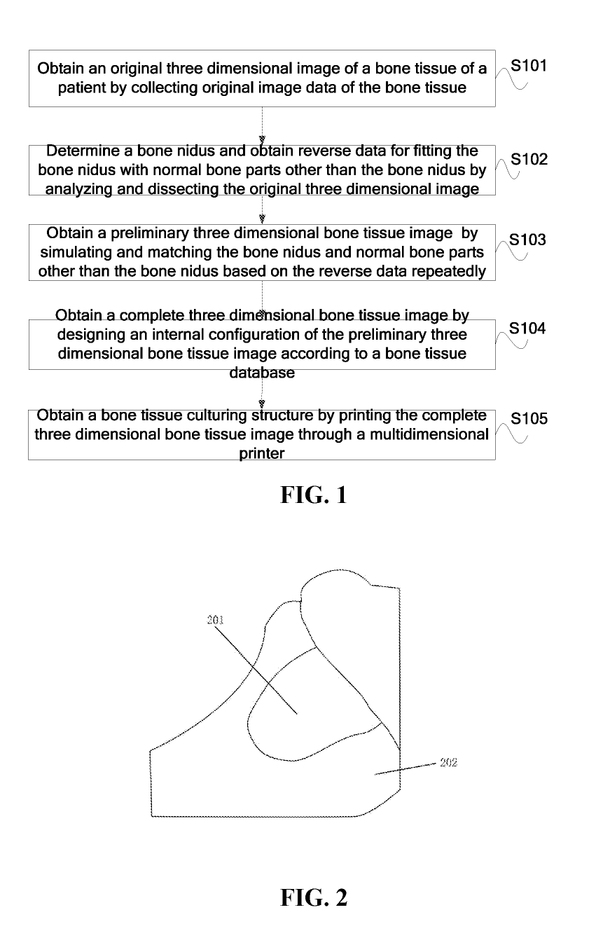

FIG. 1 is a flow chart illustrating a method for culturing osteocytes according to an embodiment of the present disclosure. As shown in FIG. 1, the method for culturing osteocytes provided in the present embodiment includes steps described below.

In S101, original image data of a bone tissue of a patient is collected, and an original three dimensional image of the bone tissue is obtained based on the original image data.

In S102, the original three dimensional image is analyzed and dissected to determine a bone nidus and obtain reverse data for fitting the bone nidus with normal bone parts other than the bone nidus.

FIG. 2 exemplarily illustrates a bone nidus 201 and a structure 202 of normal bone parts other than the bone nidus.

In S103, the bone nidus and the normal bone parts other than the bone nidus are repeatedly simulated and matched according to the reverse data, and a preliminary three dimensional bone tissue image is obtained.

Optionally, after the preliminary three dimensional bone tissue image is obtained, the method further includes converting or storing the preliminary three dimensional bone tissue image as a file of at least one of the following formats: stl, stp, obj, max, 3ds, ma, vtk and igs.

In S104, an internal configuration of the preliminary three dimensional bone tissue image is designed according to a bone tissue database to obtain a complete three dimensional bone tissue image.

Optionally, the internal configuration of the preliminary three dimensional bone tissue image is designed as follows: the internal configuration of the preliminary three dimensional bone tissue image is designed and adjusted according to conditions required for osteocyte growth and propagation.

The internal configuration of the preliminary three dimensional bone tissue image may be a porous configuration.

Optionally, the internal configuration of the preliminary three dimensional bone tissue image is designed and adjusted as follows: designing the internal configuration of the three dimensional image as a scaffold with a porous configuration; and adjusting porosity of the porous configuration, a size of aperture, a shape of a hole, surface area of the scaffold and a configuration of the scaffold.

Optionally, the shape of the hole in the porous configuration may be cylindrical, spherical or other three dimensional configurations, which are not specifically limited.

In S105, the complete three dimensional bone tissue image is printed by a multidimensional printer to obtain a bone tissue culturing structure.

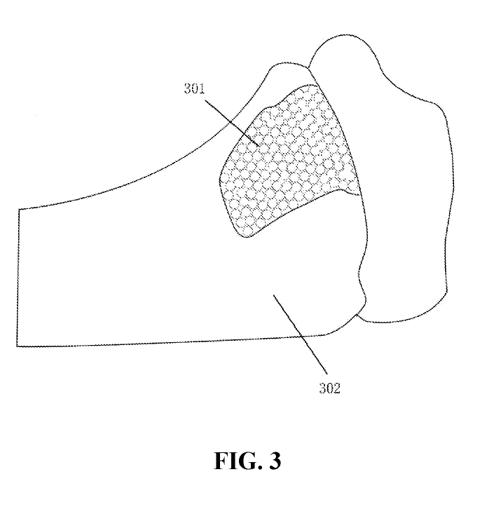

FIG. 3 is a schematic diagram in which a bone tissue culturing structure according to an embodiment of the present disclosure and remained normal bone parts of the bone tissue are matched with each other. Optionally, as shown in FIG. 3, a bone tissue culturing structure 301 is completely matched with the normal bone parts other than the bone nidus 302, and the bone tissue culturing structure 301 remedies the bone nidus. Optionally, a profile of the bone tissue of a patient when the bone tissue culturing structure 301 is matched with the normal bone parts other than the bone nidus 302 is consistent with the profile of the complete bone tissue of the patient before the part of bone nidus is damaged.

Optionally, after the bone tissue culturing structure is obtained in S105, the method further includes:

measuring and obtaining parameters of the bone tissue culturing structure; and

comparing the parameters of the bone tissue culturing structure with design parameters of a complete three dimensional bone tissue image, so as to determine whether the bone tissue culturing structure meets design requirements.

Optionally, the parameters of the bone tissue culturing structure include at least one of the followings: the porosity of the internal configuration of the bone tissue culturing structure, the size of aperture, the shape of aperture, the surface area of the scaffold and the configuration of the scaffold.

Optionally, after the bone tissue culturing structure is obtained, the method further includes: comparing the bone tissue culturing structure with a physical model of the normal bone parts other than the bone nidus to perform verification.

Optionally, after the bone tissue culturing structure is obtained, the method further includes: determining whether the bone tissue culturing structure, which is served as a microenvironment structural body, meets the conditions for osteocyte growth and propagation.

Optionally, the bone tissue culturing structure is a physical structure matched with the normal bone parts other than the bone nidus.

Optionally, after the complete three dimensional bone tissue image is obtained, the method further includes converting or storing the complete three dimensional bone tissue image as a file of at least one of the following formats: stl, stp, obj, max, 3ds, ma, vtk and igs.

Optionally, the multidimensional printer includes: a three dimensional (3D) printer, a four dimensional printer or a five dimensional printer.

The method for culturing osteocytes provided in embodiments of the present disclosure designs a bone tissue culturing structure which is suitable for osteocyte growth. The configuration of the bone tissue culturing structure is adjustable. Individual bone data (the normal bone parts other than the bone nidus of the patient) is matched with the data in the bone tissue database, and the bone tissue culturing structure and the patient can be subjected to individualized matching. The quantity, sizes, distribution and shapes of micropores in the internal configuration of the bone tissue culturing structure can be manually controlled. The osteocytes can be accurately positioned while the bone tissue culturing structure is built. The osteocytes can be contained on the surface and in the bone scaffold, thereby providing a possibility of realizing reasonable spatial distribution of the osteocytes; conduit structures similar to blood vessels in natural bone tissue can be built; and conduit distribution, conduit diameters and porosity can be manually controlled to facilitate exchange of oxygen, nutrients and metabolites between the osteocytes in the bone tissue culturing structure and an external environment and establish a basis for further culturing and forming blood vessel structures and even finally producing corresponding physiological functions.

The bone tissue culturing structure suitable for osteocyte growth and propagation, which is printed by a multidimensional printer, has a set of mutually communicated channel systems, so that the cells and the nutrients are smoothly transported into the tissue and organ structure in a rebuilding process of the tissue and organ, and the cells are uniformly deposited into a microchannel.

The above only describes an embodiment of the present disclosure, not intended to limit the present disclosure. Technical solutions obtained by adopting equivalent replacements or equivalent transformations fall within a protection scope of the present disclosure.

### INDUSTRIAL APPLICABLITY

The bone tissue culturing structure, which is prepared by the method for culturing osteocytes provided in embodiments of the present disclosure, is suitable for cell growth and propagation, and can provide a proper microenvironment for forming a bone tissue that remedies the bone nidus.

1. A method for culturing osteocytes, comprising:
obtaining an original three dimensional image of a bone tissue of a patient by collecting original image data of the bone tissue;
determining a bone nidus and obtaining reverse data for fitting the bone nidus with normal bone parts other than the bone nidus by analyzing and dissecting the original three dimensional image;
obtaining a preliminary three dimensional bone tissue image by simulating and matching the bone nidus and the normal bone parts other than the bone nidus based on the reverse data repeatedly;
obtaining a complete three dimensional bone tissue image by designing an internal configuration of the preliminary three dimensional bone tissue image according to a bone tissue database; and
obtaining a bone tissue culturing structure by printing the complete three dimensional bone tissue image through a multidimensional printer.

2. The method for culturing osteocytes according to claim 1, wherein the designing an internal configuration of the preliminary three dimensional bone tissue image comprises: designing and adjusting the internal configuration of the preliminary three dimensional bone tissue image according to conditions required for osteocyte growth and propagation.

3. The method for culturing osteocytes according to claim 2, wherein the designing and adjusting the internal configuration of the preliminary three dimensional bone tissue image comprises:
designing the internal configuration of the preliminary three dimensional bone tissue image as a scaffold with a porous configuration; and
adjusting porosity of the porous configuration, a size of aperture, a shape of a hole, surface area of the scaffold and a configuration of the scaffold.

4. The method for culturing osteocytes according to claim 3, wherein the shape of the hole in the porous configuration is cylindrical shape or spherical shape.

5. The method for culturing osteocytes according to claim 1, wherein the method further comprises the following steps after the obtaining a bone tissue culturing structure:
measuring and obtaining parameters of the bone tissue culturing structure; and
comparing the parameters of the bone tissue culturing structure with design parameters of the complete three dimensional bone tissue image to determine whether the bone tissue culturing structure meets design requirements.

6. The method for culturing osteocytes according to claim 5, wherein the parameters of the bone tissue culturing structure comprise at least one of the followings: a porosity of the internal configuration of the bone tissue culturing structure, a size of aperture, a shape of aperture, surface area of the scaffold and a configuration of the scaffold.

7. The method for culturing osteocytes according to claim 1, wherein the method further comprises the following step after the obtaining a bone tissue culturing structure:
comparing the bone tissue culturing structure with a physical model of the normal bone parts other than the bone nidus to perform verification.

8. The method for culturing osteocytes according to claim 1, wherein the method further comprises the following step after the obtaining a bone tissue culturing structure: detecting whether the bone tissue culturing structure served as a microenvironment structural body meets conditions required for osteocyte growth and propagation.

9. The method for culturing osteocytes according to claim 1, wherein the bone tissue culturing structure is a physical structure matched with the normal bone parts other than the bone nidus.

10. The method for culturing osteocytes according to claim 1, wherein the method further comprises the following step after the obtaining a preliminary three dimensional bone tissue image: converting or storing the preliminary three dimensional bone tissue image as a file of at least one of the following formats: stl, stp, obj, max, 3ds, ma, vtk and igs.

11. The method for culturing osteocytes according to claim 1, wherein the method further comprises the following step after the obtaining a complete three dimensional bone tissue image: converting or storing the complete three dimensional bone tissue image as a file of at least one of the following formats: stl, stp, obj, max, 3ds, ma, vtk and igs.

12. The method for culturing osteocytes according to claim 1, wherein the multidimensional printer comprises at least one of the followings: a three dimensional printer, a four dimensional printer or a five dimensional printer.
